(19) United States
(12) Patent Application Publication
(10) Pub. No.: US 2026/0341428 A1
(43) Pub. Date: Oct. 29, 2026

(54) KIT FOR USE IN CONDUCTING BLINDED N-OF-1 TRIALS

(71) Applicant: ITrial.me Inc., Andover, MN (US)

(72) Inventors: Eric L. Granstrom, Andover, MN (US); Joseph Tor-Bjorn Masrud, Mahtomedi, MN (US)

(21) Appl. No.: 19/737,244

(22) Filed: Jul. 10, 2026

Related U.S. Application Data
(63) Continuation of application No. 18/775,389, filed on Jul. 17, 2024.
(60) Provisional application No. 63/516,039, filed on Jul. 27, 2023.

(51) Int. Cl. G16H 10/20; A61B 5/00; G16H 10/60; G16H 15/00; G16H 20/00; G16H 40/40; G16H 40/67

(52) U.S. Cl. CPC G16H 10/20; A61B 5/4848; G16H 10/60; G16H 15/00; G16H 20/00; G16H 40/40; G16H 40/67

(57) ABSTRACT

Disclosed are kits for use in conducting blinded N-of-1 trials to safely and reliably determine effectiveness of treatments for individuals. As such, these kits are useful in testing the efficacy and effectiveness of substances such as, for example, nutritional supplements, in a personalized manner with respect to a specific subject, and further relate to tailoring prescribed treatments, supplements, prescriptions, or medicines for that specific subject. This may involve designing, customizing, facilitating, analyzing, and reporting personalized research in which participants are assisted in self-administering randomized controlled trials of interventions.

### RELATED APPLICATIONS

This application is a continuation of U.S. patent application Ser. No. 18/775,389, filed Jul. 17, 2024, which claims priority to U.S. Provisional Patent Application Ser. No. 63/516,039, entitled “METHOD TO DETERMINE THE EFFICACY OR EFFECTIVENESS OF A SUBSTANCE” and filed Jul. 27, 2023, the entire contents of which are hereby incorporated by reference.

### TECHNICAL FIELD

The present disclosure generally relates to methods of performing health care treatment research, including personalized research.

### SUMMARY

In accordance with certain aspects, the present disclosure describes systems and methods in which participants are provided with assistance in self-administration of randomized controlled trials that measure a treatment effectiveness against a control (placebo or another treatment) for a participant. Such methods and systems simultaneously utilize standardized and customizable yet highly automatable processes that provide assistance in electronic format as well as in physical materials.

In certain aspects, the present disclosure includes a method in which consumers direct the purchase of assistance in performing a rigorous N-of-1 clinical trial upon themselves. This may be accomplished, for example, through an online website, portal, or software application on a smartphone, tablet, computer or other device. The consumer navigates through a list of pre-constructed and/or adaptive protocols appropriate to a variety of possible interventions (or designs and uses their own protocol), thereby providing the consumer the ability to discretionarily choose and possibly customize interventions and outcomes pertaining to their specific health interests. The method subsequently provides an individual with access to blinded interventions (that is, one treatment's effect relative to that of another, or one treatment's effect relative to those of a control, placebo, or sham condition) at a location of the individual's choosing such that the individual can remain unaware throughout the trial whether, for instance, future specific conditions of a trial represent an active treatment or a control. The consumer may also be provided with access to personalized progress and account information on a remote platform (web, smartphone application, and so forth) that also provides assistance in the form of prompts and schedules for dosing and outcome measurements. The consumer may also be provided with an automated statistical analysis suite to calculate the results of the trial and communicate trial results to the consumer and, if desired, their health care team. Methods and systems in accordance with various aspects of the present disclosure provide assistance to the participant at various stages, allowing the participant to administer an N-of-1 trial upon themselves. In certain aspects, the trial continues until the earliest of participant chosen termination, scheduled termination, interim data analysis triggered termination, and so forth.

In certain aspects, methods include providing a consumer with options for various dietary supplements (materials, quantities, concentrations, form factors), trial durations, periods between dosing, statistical confidence levels, health outcomes, and so forth. In certain aspects, such options and protocols for providing have been assessed by a trial service provider as viable experimental configurations. In certain aspects, methods provide for treatments directed to formal medical conditions or disease states, alternatively methods provide for health/wellness outcomes, dietary outcomes, or athletic/performance outcomes.

In certain aspects, methods include shipping intervention (treatment) materials such as medical devices, dietary supplements, nutraceuticals, pharmaceuticals, or the like, to a consumer's home or selected address, and performing a trial in a fashion referred to as a “decentralized” format, or a “virtual” trial in which no face-to-face meetings with service providers are required, and likewise no travel of the consumer away from their home may be required.

In certain aspects, methods include providing for self-administration of N-of-1 trials for testing of a supplement such as melatonin as a sleep aid, or for testing a supplement such as fish oil to assist with joint pain, or for testing a supplement such as vitamin B12 to assist with mental focus or clarity.

In certain aspects, methods include providing for consumers to choose outcomes unspecified by a trial service provider or to choose outcomes specified by the consumer themselves. In the case of a sleep trial, a pre-validated outcome measure may be provided by the trial service provider, such as a standard sleep index instrument, such as the Pittsburgh Sleep Quality Index (PSQI), or any of multiple instruments developed by the National Sleep Foundation such as the Sleep Satisfaction Tool. Alternatively, a trial service provider might offer a consumer the prospect of providing their own outcome metric, perhaps in the form of a “daily question” that the automated process would prompt the participant to answer periodically in accordance with a scoring scale such as a traditional Likert scale having numeric choices that range, for instance, from 1 through 5.

In certain aspects, methods include providing a statistical analysis to estimate an effect size and a confidence interval in an effect size. Note that an effect size may be measured as a difference between two different conditions (for example, active treatment versus control, or one active treatment versus another active treatment). Though the estimate may thus be in the form of an effect size difference, for ease of description, the present disclosure may refer to it as simply an effect size.

In certain aspects, methods include providing a statistical analysis to estimate likelihood that an effect size is larger than (or smaller than) zero or alternatively larger than (or smaller than) a critical threshold such as a minimum effect size of perceived consequence to a participant. In certain aspects, methods include providing a statistical analysis that is performed numerous times during various points in time during the trial. In certain aspects, methods include providing or displaying trial results to the participant graphically.

In certain aspects, methods include displaying trial results of other participants of previous and/or similar trials to the participant. This may be prior to a trial to assist the participant in customizing their own trial, or may be after a trial for the purpose of comparing the participant's individualized results with results from other members of a broader population or series of subpopulations of a broader population (for example, comparison against all participants in similar trials, or comparison against participants of the same gender).

In certain aspects, methods include providing an outcome measurement system that may be digital, electronic, and/or automated, such as a digital sleep tracker. In certain aspects, an outcome measurement system may use a mixture of automated and manual outcome monitoring.

In certain aspects, methods include providing for a reduction in price and/or other incentives to a consumer for completion and/or compliant self-administration of specific trials, for instance those of greater use or interest to a supplier of supplements or trial service provider. Incentives may be provided to a consumer for completion of trials with high treatment and/or outcome reporting compliance. Incentives may be provided for consumers with specific demographic or medical characteristics, providing information about historical supplement use, or for simply providing thorough demographic or medical information to the trial service provider.

In certain aspects, methods include prompting or suggesting that a consumer consider an additional trial. For example, after the success or failure of a trial (or potentially during a trial that has intermediate results pointing to failure or success) a consumer may be prompted to conduct another trial at a different dosage (lower or higher) or with an alternative intervention. Consumers may also be prompted to purchase access to the treatment itself outside of the trial, for example a one-time purchase of supplements or a subscription to an ongoing stream of supplements. Consumers may also be prompted to considering quitting a trial.

In certain aspects, methods include appending the results of a trial to a database of similar trials, and/or, when appropriate, including the results of a trial in an updated webpage presentation of results to the public, in a scientific analysis and/or publication in scientific journals, and/or in a submission to the FDA or other regulatory body for request for approval, and so forth.

In accordance with various aspects, the present disclosure describes a system for providing self-administrable clinical trial to determine effectiveness of a treatment on an individual. Such a system may include an information module configured to provide the individual with access to information about customizable clinical trials and available treatments for testing, a customization module accessible by the individual and configured to allow selection of trial parameters in accordance with a trial protocol architecture, and an ordering module configured to facilitate completion of an order by the individual, and to order blinded packaging of treatment and control materials for delivery to the individual. The system may also include a user interface configured to prompt the individual to comply with a treatment regimen of the treatment and control materials, and to facilitate recording of outcome measures by the individual. The system may further include a statistical analysis module configured to calculate results of the trial based on outcome measures recorded by the individual, and to communicate the results to the individual. In certain aspects, the information about customizable clinical trials and available treatments for testing includes results of previous tests.

In accordance with certain aspects, the present disclosure provides for “peeking” at interim results during a clinical trial performed according to various embodiments disclosed herein. Peeking at interim results may be responsive to consumer preference, to provide for early termination of successful (or unsuccessful) tests, to allow for adjustments or modifications of the trial to be made, and so forth. Peeking processes may involve randomizing trial period sequencing, supplying blinded treatments, and packaging of blinded treatments such that any knowledge of the ingredients of one package would be non-predictive of the ingredients of any other package. Interim results may be communicated to the individual or others without adversely affecting future treatment blinding.

In accordance with certain aspects, the present disclosure provides for moving baseline trials. For example, when carryover effects from one treatment period to the next are non-negligible, the carryover effect sizes and durations may be mathematically estimated in the course of generating an overall treatment effect estimate. In accordance with certain aspects, the evolving heath/wellness states of a trial participant under such conditions may be accommodated by fitting the shape of each new outcome response curve over time by varying treatment condition. As such, trials can continue to be performed on moving baseline conditions utilizing newly acquired data. Overall, such moving baseline trials may proceed similarly to trials conducted in accordance with various embodiments disclosed herein, including using a trial service provider to assist in performing self-administered clinical trials, customizing trial parameters, receiving prompts for treatments and outcome measure recording, and providing statistical results including effectiveness estimates.

In accordance with certain aspects, methods of the present disclosure may be used to estimate carryover periods. For example, a dosing sequence may be randomized, and the repeated dose events can be isolated from a data analysis perspective without unblinding or revealing anything to the participant, to thereby better estimate steady state effectiveness from the measurements made during such repeated dose events. Learnings from such carryover period estimation may be used to adapt trial parameters during the trial.

The details of one or more aspects of the disclosure are set forth in the accompanying drawings and the description below. Other features, objects, and advantages of the techniques described in this disclosure will be apparent from the description and drawings, and from the claims.

### BRIEF DESCRIPTION OF THE DRAWINGS

The description and claims will be better understood in context with the following figures which are offered as examples but not intended to be exhaustive nor complete detailing of the invention.

FIGS. 1A-1D are flow charts illustrative of certain aspects of methods in accordance with the present disclosure.

FIG. 2 is a flow chart illustrating peeking at results during a trial while maintaining blinding in accordance with certain aspects of the present disclosure.

FIG. 3 is a schematic depiction of steps that may be included when estimating effectiveness in certain aspects of the present disclosure.

FIG. 4 is a flow chart illustrating in-trial parameter adjustment in accordance with certain aspects of the present disclosure.

FIG. 5 is a schematic depiction of steps that may be included in trials having moving baselines in accordance with certain aspects of the present disclosure.

### DETAILED DESCRIPTION

The present disclosure relates to testing the efficacy and/or effectiveness of a substance, material, or condition, such as, for example, a nutritional supplement, in a personalized manner with respect to a specific subject, and further relates to tailoring a prescribed treatment, supplement, prescription, medicine, or device for that specific subject. This may involve designing, customizing, facilitating, analyzing, and reporting personalized research in which participants are assisted in self-administering randomized controlled trials of interventions. Such methods make simultaneous use of standardized procedures as well as customizable and automated processes to providing assistance to participants in their self-administration of controlled trials related to the efficacy or effectiveness of a substance, including through the provision of information, the arrangement for placebos indistinguishable from the substance, maintaining the blinding of the trial and allocation concealment, customizing and optionally adapting or adjusting the trial, the provision of a user interface and portal for providing response and retrieving results, and providing personalized analysis, results, recommendations, and/or incentives that may be based on inferential statistics as well as aggregated data from other participants.

Methodologies in accordance with the present disclosure may be used to determine effectiveness or efficacy of a treatment on an individual, as opposed to on a population. Generally, effectiveness relates to the performance of a treatment under real world conditions, whereas efficacy relates to the performance of a treatment under idealized conditions. Aspects of the present disclosure may be used for either or for both. Moreover, it should be noted that effectiveness and efficacy of a treatment are determined in relation to a control, which may be a placebo, a different treatment, a different dose of the same treatment, and so forth. It will be understood that aspects of the present disclosure apply to all of these, regardless of whether a specific example (such as effectiveness versus placebo) is being used to illustrate.

Personalized medicine increasingly demonstrates the power of tailoring a treatment to a specific subject. Genes, bodies and body types, diets, lifestyles, and so forth, all render an individual patient quite unique with respect to any possible treatment plan as well as for any possible desired health outcome. As such, the most readily applicable evidentiary data, even beyond large population randomized controlled trial data, may come from “N-of-1” trials in which an individual patient is tested in accordance with a time-alternating and, ideally, blinded sequence of a control (typically a placebo or a “sham” treatment) and an active treatment (often referred to just as a treatment), with the effects of the active treatment capable of being isolated against a control and optimized for that individual and their unique condition and goals. However, it would be difficult and highly impractical, if not impossible, for N-of-1 trials to be assessed, selected, and administered, for instance, through the existing network of approximately 900,000 practicing U.S. medical doctors choosing from among 50,000 existing pharmaceutical options for studying optimal use for the health of 300,000,000 U.S. patients in a virtually unlimited series of individual N-of-1 tests, particularly with such a network lacking the knowledge, specialization, protocols, capacity, and statistics to perform such a new service.

Thus, in accordance with the present disclosure, systems and methods are provided that allow N-of-1 trials to be accessible, customizable, self-administrable, adjustable, affordable, and ultimately useful in allowing individual patients (also referred to as customers, participants, and so forth) to find out what works for them, largely on their own. Moreover, it will be recognized that the systems and methods described herein are equally applicable for determining if a treatment doesn't work (for example, a consumer may be currently taking a treatment and they want to determine whether and to what extent it is actually helping them in the manner that they would like).

The gold standard for modern scientific research in health care treatments is the randomized controlled trial (RCT). RCTs are prospective studies in which participants are randomly allocated to one or more interventions or control conditions (placebos) and monitored for one or more outcomes. RCTs are used broadly to estimate the effectiveness or efficacy of an intervention relative to a control condition (or two or more treatments). The most traditional RCT is a large-population, parallel (non-crossover) RCT, which is highly effective at estimating an effect size as averaged across a broad participant population. However, in non-crossover RCTs, because no participants are subjected to both the experimental intervention and the control conditions, such traditional trials are incapable of estimating effect sizes of any specific individual within the trial. In other words, traditional RCTs seek to determine an average effect on a member of a population, not the specific effect on any individual.

N-of-1 trials are multiple-crossover, individual participant RCTs that represent a relatively new and underused trial format capable of effect size estimation for an individual. In N-of-1 trials, rather than having two parallel groups exposed to experimental intervention (for example, group A) and control (for example, group B), a single individual is subjected to intervention and control conditions at varying points in time, for instance in an ABAB sequence. The individual may be allocated to a specific sequence (for example, ABAB rather than ABBA, BAAB, or BABA), and have that allocation concealed to them. Further, they may be blinded, meaning that after they are randomly assigned to that sequence, they remain unaware of which specific sequence they have been assigned. Further, a trial may be double or triple blinded, where beyond individual participant unawareness of treatment sequence, so too may a tester or statistical analyst remain unaware of the treatment sequence. Comparison of outcomes across those different periods allows effect size estimation that is specific to the individual.

While N-of-1 trials offer advantages such as individualized, relevant results, they are relatively rarely used. Of the approximately 500,000 clinical trials registered over the past two decades with the U.S. National Institute of Health's ClinicalTrials.gov trial tracking system, the number of formal N-of-1 trials performed worldwide appears dramatically smaller, perhaps on the order of hundreds per year. The relative disuse of N-of-1 trials may be attributable to various factors. First, they are simply unknown to many researchers, care providers, and potential participants. Further, typical research focuses on treatments of prospective use to broad populations, whereas the structure of an N-of-1 trial is designed to find responses for an individual (that is, to isolate heterogeneity in patient treatment response). Due in part to their lack of use, N-of-1 trials do not have nearly the same institutional systems and infrastructure developed to provide and support them as compared to traditional RCTs. Moreover, N-of-1 trials are considered substantially more expensive per participant.

Although an estimate of an average effect size across a large population may be of some general interest to an individual who is considering an intervention such as taking a supplement, treatment, or medicine, the question of more immediate consequence is whether the intervention will be effective for them specifically, not “on average.” The value of individualized medical research information is potentially very high while remaining unavailable by traditional research means. A participant seeking to know treatment effectiveness, for instance, for melatonin as a sleep aid on themselves can review general (large population trial) literature, but has no current mechanism by which to have an N-of-1 trial performed on themselves. Melatonin, though ubiquitously available in retail stores across the U.S. as a dietary supplement, is not available commercially in N-of-1 clinical trial form. Moreover, primary care health care providers would not be in a position to assist an individual in conducting an N-of-1 trial, and academic researchers similarly aren't situated to provide one-person trials of a participant's choosing. Further, should an individual wish to perform a trial independently, they would struggle with trial protocol design, placebos, self-blinding, and statistical interpretation. These are among the factors that make N-of-1 trials and the resulting data inaccessible.

The inaccessibility of N-of-1 trials is unfortunate given that the results could be critical in optimizing individual health outcomes. As an example, the relatively common drug codeine is now known, only after extensive research, to provide effectively no analgesic benefit in approximately 25% of the population due to specific genetic differences. A large population study may show, on average, codeine to be very effective, and, in isolation, prompt a health care provider to conclude it to be a useful analgesic for their clientele. Unfortunately, about 25% of individuals relying on it for pain relief would have extremely disappointing results. The lack of personalized medical data knowledge across a litany of treatments for a litany of outcomes represents a gap in modern health care, and there is currently no methodology established for closing that gap. Various aspects of the present disclosure are presented to address this issue by providing a protocol for designing individualized N-of-1 trials, a method for self-administering and for ensuring compliance with the trial regimen, and a way to collect relevant data and deliver individualized results. Benefits include not only confirming health benefits in some treatments, but also in failing to see benefits (that is, disproving effectiveness) in other treatments (and/or seeing detrimental outcomes).

In the present disclosure, it is recognized that funding has been a barrier for the performance of N-of-1 trials. In traditional research contexts, the per-participant cost of N-of-1 trials may be expected to be much larger than for traditional (parallel) RCTs. Moreover, traditional RCTs enjoy traditional funding sources seeking data on large populations for the purpose of regulatory clearance of potentially lucrative novel treatments. As such, various aspects of the present disclosure are directed to models whereby the individual participants of N-of-1 trials, being those who both perceive and realize the greatest direct benefit of individualized results, are the direct customers. Aspects of such models may also include various incentives, cost breaks, and assistance directed to furthering the reliability of the study, the compliance of the participant, and the applicability of the resulting data.

As recognized in the present disclosure, inferential statistics may be used to allow interim results to be revealed directly to participants during an N-of-1 trial, and not just revealed to a third party (for example, a Data and Safety Monitoring Board), without invalidating results or impeding statistical rigor in hypothesis testing. In accordance with various aspects of the present disclosure, procedures are provided that allow interim results to be revealed while maintaining allocation concealment and/or participant blinding to the future dosing sequence in a specific N-of-1 trial. Reporting interim results to participants may dramatically increase participant interest in and use of trial services, particularly if methodological rigor (including blinding) can be maintained.

The present disclosure thus identifies key gaps in N-of-1 method development that adversely affect their availability to individuals. Recognizing that individual customers will need unique solutions, both because of heterogeneous treatment results as well as heterogeneous treatment choices, consumers will be demanding of unique services tailored to their desires. Thus, standardization is needed (to maintain broad accessibility and low cost) simultaneously in concert with high customizability, which presents a non-traditional pairing. Moreover, to minimize costs and maximize access and use, trials should be self-executable by lay users with assistance from automated systems, thereby resulting in a certain amount of disintermediation of traditional medical service providers such as retail pharmacists and primary care practitioners. In addition, the supporting methods would benefit from broad (multi-modal) method innovation, whereby a process can assist not just with digital techniques (for example, statistical analysis) but also with practical challenges such as arranging for placebos and blinding of a participant while maintaining the participant's ability to assign outcomes to the appropriate treatment.

The value to an individual of knowing the effectiveness of a treatment specifically for them (as opposed or in addition to for a population) may dramatically exceed the cost of the treatment itself. For instance, in the case of a litany of dietary supplements, the cost of treatments may be relatively low, for example on the order of $0.01 per daily dose. However, the value of a successful treatment may be multiple orders of magnitude higher, even when simply assisting in areas of general health and wellness outcomes such as temporary relief of minor pain, assistance in daily sleep, reducing fatigue, gaining energy, improving cognition and mental acuity, and so forth.

Rigorous scientific data on effectiveness for many substances, and in particular for a variety of materials referred to in the U.S. as “dietary supplements” or simply “supplements,” is particularly limited, and what data does exist is usually related to estimated average difference between populations receiving a treatment or a control, and not the individual effect for a specific potential consumer. Existing methods to provide potential consumers rigorous clinical trial data assessing effectiveness of a treatment on that potential consumer are thus limited. Conceptually, execution of a N-of-1 trial on an individual can provide such data, but existing methodologies are not adapted for, nor available to, individual consumers. Traditional academic researchers performing RCTs on specific topics of interest to those researchers (typically regarding novel treatments) are ill-equipped to handle the general public's interest in a diversity of treatments. Nor are traditional academic researchers equipped to handle consumer expectations with respect to trial time frames or costs. Lay individuals lack the training to devise appropriate protocols or statistics to analyze results, not to mention the challenges presented in preparation of placebos and maintaining blinding.

Alternative commercial trial service providers also do not offer methodologies that are sufficiently consumer-centric to allow for rapid scaling, adoption, and ubiquitous use. Each of the following limits method versatility: reliance on centralized trials; patient needs to coordinate a trial between a health care practitioner, trial service provider, and pharmacist; travel to care centers and pharmacies; lack of opportunity for a consumer to customize trials to fit their specific desires (such as duration and statistical confidence); timeliness; lack of interim data analysis or visibility to consumer or allowing consumer to choose early trial termination based on interim results; lack of blinding; cost; lack of adaptation during a trial to improve trial design based on interim results; lack of routine consumer prompting or compliance oversight for dosing and outcome reporting during trial execution; and lack of ability to use multiple-crossover-trials on treatments having long or unknown carryover periods.

Accordingly, disclosed herein are methods of performing clinical trials using N-of-1 research methods and crossover designs that are tailored for individualized consumer purposes. Such methods may be used in the performance of contracted materials supply and corresponding blinding services of N-of-1 trials of treatments (such as supplements) that are available commercially to the paying public with the intent of assisting individuals in determining whether a treatment is meeting their personalized goals. Also disclosed herein are methods of performing such services, particularly in fashions that are unanticipated and new relative to the prevailing practice of clinical trials.

In the present disclosure, it is recognized that the logistical burden of generating and implementing a unique protocol, Institutional Review Board (IRB) approval, and so forth, for each customized N-of-1 design could be prohibitive in traditional trial strategies. As such, the present disclosure provides for erecting a broad, or “umbrella,” protocol structure within which a large array of individual clinical processes can be used. In certain instances, a single protocol and IRB approval can be used to cover not just multiple patients, multiple supplements, multiple anticipated health outcomes, multiple durations, and so forth, but an indefinite number of each. In accordance with various aspects of the present disclosure, a protocol architecture may be applied across a litany of processes, particularly in cases when the safety of the supplement or other material being tested is relatively high, the labelled use conditions are pre-existing, and experimentation can be described that is within the existing bounds of known safe use.

Accordingly, an overarching protocol architecture (which may include an IRB approval) may define an umbrella protocol within which an entire family of unique processes may be established. Within such a clinical trial space, further improvements to more traditional trial methods may be available. For instance, in a traditional trial a responsible party imposes an intended or targeted health outcome on a patient. In accordance with the present disclosure, a commercial supplier or a clinical trial service may generate and present an array of health outcome options for the participant to choose from, and/or health outcomes may be entirely participant-defined. For instance, a customer may be asked to build their own outcome metric scale. (“Write a question that you would like to periodically have asked about some element of your health or wellness during the trial. Identify what a score of 1 would mean on that scale, as well as a score of 3, 5, 7, and 9. Attempt to pick a scale that is likely to have some days earning a score below 3 and some days scoring above 7 to help with subsequent analysis. Also, if possible, find ways to clearly define what those scores would be in objective or quantifiable terms.”) Multiple outcomes may be defined, measured, and weighted according to participant goals. Outcomes to be tracked may be anticipated as traditional, positive health/wellness outcomes (for example, hours of daily sleep). Tracked outcomes may also be negative, such as “side effects” or adverse events.

A potential benefit of using pre-existing or anticipated sets of outcomes is that they are more likely to match those used by many other customers in other trials testing the same supplement (or other treatment) and using a similar set of test procedures. In such circumstances, the results of those tests may be more amenable to aggregation and meta-analysis along with other similar data, making the results more useful for publication or use as a larger population clinical trial. In that case, there may be added value to the public as well as to the commercial seller of trials or supplements (for instance, data applicable to a large population may help validate an anticipated improvement along a specific vector of general health or wellness, and that extra validation may make it easier to market the use of that supplement to future users). The supplier may therefore wish to incent patient choice of a validated/standardized (common) health outcome variable to be tracked instead of (or in addition to) a customized or patient-defined outcome. Dynamic pricing and/or refund structures may be offered whereby, within a pay-for-treatment N-of-1 supplement model, payments may be reduced (subsidized, reimbursed, credited for future purchase, and so on) for participants who select treatment protocols and outcome metrics that facilitate subsequent data use or meta-analysis by other parties.

In addition to automated and prompted changes in test protocol, systems and methods of the present disclosure may allow a customer to choose (whether with a reason or at a whim) to change the process. As an example, a customer who initially designed a trial to last for a full year may shorten the duration part way through the trial. In such a case, the system may prompt the patient (for example, after a patient-driven change request) regarding how the process may be changed mid-experiment and what impacts such a change would have on statistical validity. In many traditional tests, noncompliance with an original intent-to-treat would dramatically impair not only the validity of that individual's results within a broader trial, but also impair the broader large population trial. However, in accordance with the present disclosure, trial validity (both individually, and in a large-population or meta-analysis context) may be maintained during a mid-trial change or adaptation. Such adaptations include dynamically using the results of other patient data during a trial to update experimental parameters of a current or future customer's trial. Adaptations may also include changing dosing amount during the trial. This may be facilitated by providing smaller dose pills (for example, 5 mg rather than 10 mg or 20 mg), so that the dosing can easily be stepped up or stepped down simply by taking more or fewer pills at each dosing cycle.

In certain aspects, methods of the present disclosure recognize and take into account situations in which the use of a placebo has a positive effect or favorable response in a blinded experiment. Whereas traditional trials involve unblinding, and in particular revealing the placebo at the end of a customer-paid-for trial, the present disclosure provides for presenting the patient (before a test) with the option of maintaining blinding and allocation concealment at the end of the test in case a positive placebo effect is obtained.

In accordance with certain aspects, N-of-1 trials may be performed as a service for supplement retailers, manufacturers or other suppliers of health-related products, for example as a means of helping them “validate” the effectiveness of their materials with data and not just bare customer testimonials.

Moreover, aspects of the present disclosure include the customization of a trial service, allowing consumers to select the treatments to be tested, regardless of manufacturer or suppliers, and have those treatments supplied to the trial-service provider for repackaging as part of a trial service.

Moreover, aspects of the present disclosure include the customization of a blinding service, allowing consumers to select the treatments to be tested, regardless of manufacturer, and supplied back to the consumer with randomization, anonymization, blinding and/or allocation concealment.

In yet further aspects, the present disclosure provides for “crowdsourcing” a choice of supplement to be offered in a blinded N-of-1 service by allowing customers pre-pay or pre-commit to pay for the development and availability of a test on such supplement.

While the various aspects of the present disclosure are applicable to a variety of conditions that may be treated by certain supplements, medicines, and medical devices, it may be useful to describe the various aspects in reference to specific conditions such as sleep difficulties, joint pain, and mental focus or clarity. It will be recognized that these are non-limiting examples.

Reference will now be made to the drawings, which depict one or more aspects described in this disclosure. However, it will be understood that other aspects not depicted in the drawings fall within the scope of this disclosure. Like numbers used in the figures refer to like components, steps, and the like. However, it will be understood that the use of a reference character to refer to an element in a given figure is not intended to limit the element in another figure labeled with the same reference character. In addition, the use of different reference characters to refer to elements in different figures is not intended to indicate that the differently referenced elements cannot be the same or similar. It will also be appreciated that the drawings are meant to illustrate certain aspects and arrangements of features in a way that contributes to their understanding and are not meant to be scale drawings that accurately represent size or shape of elements.

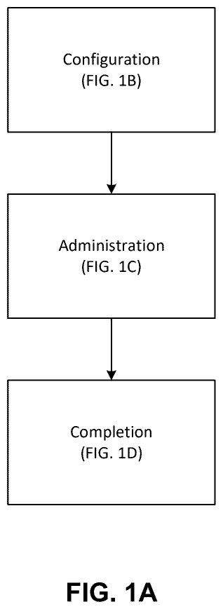

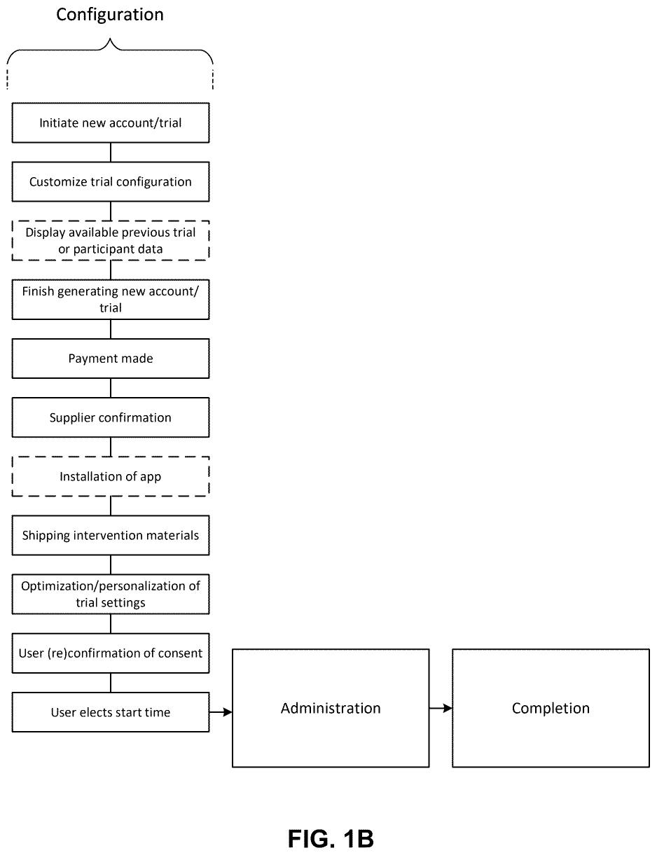

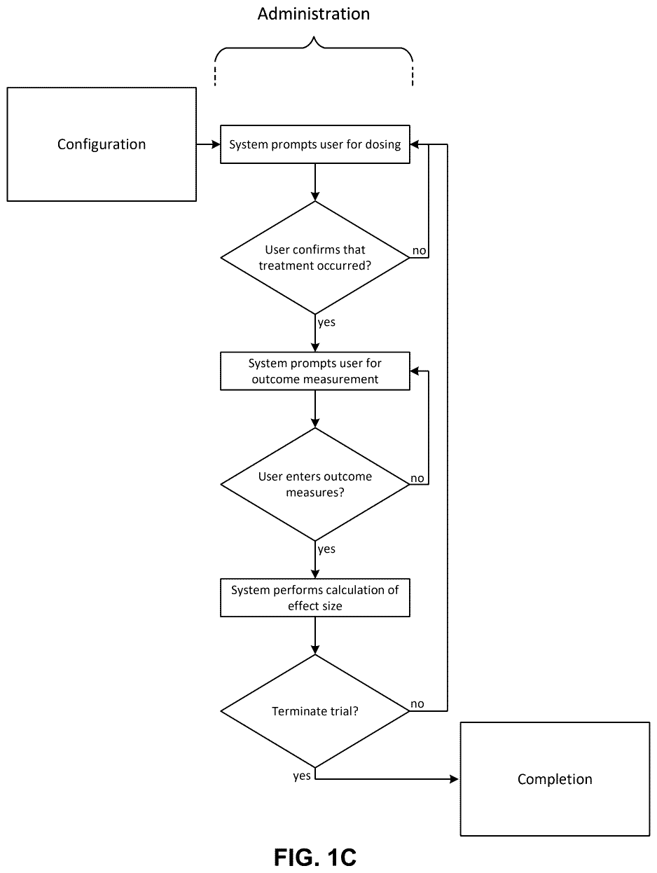

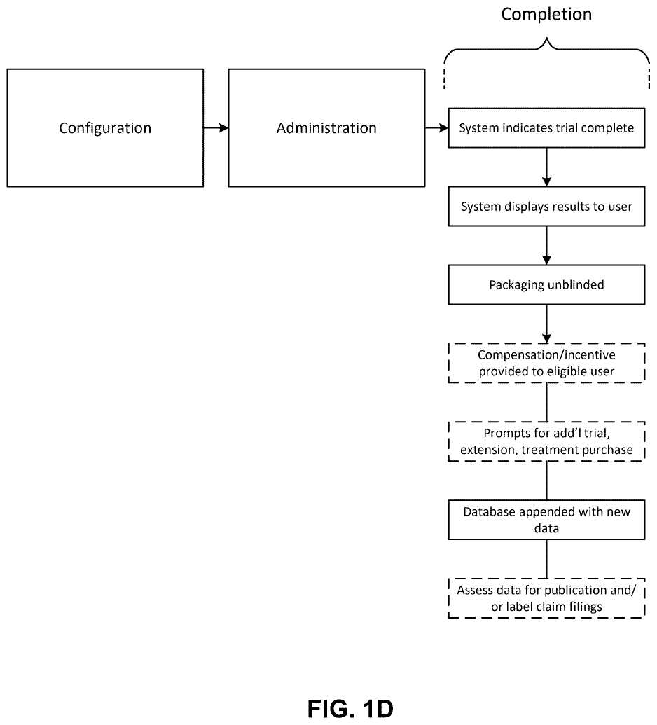

FIG. 1A shows a simplified flow chart depicting the main stages that may be involved in methodologies according to aspects of the present disclosure. Generally, a trial is configured and customized during a Configuration stage, followed by conducting the customized trial under an Administration stage, and then generating and delivering results in a Completion stage after terminating the trial. Aspects of the initial Configuration stage are further illustrated in FIG. 1B, whereas aspects of the Administration stage and Completion stage are further illustrated in FIGS. 1C and 1D, respectively.

Starting with Configuration, FIG. 1B depicts a number of steps that can be performed in the trial configuration stage. Some of these steps may be optional, and many of the steps may be performed in various orders, concurrently with other steps, and/or left incomplete while other steps are being completed. As such, there are no arrows between steps in the Configuration stage to indicate any particular required work flow, even though it may be preferable to have the steps performed roughly in the order shown. Also, there may be additional steps not shown or that are included implicitly (or otherwise) in the performance of other steps that are shown.

In general, the Configuration stage begins with a consumer (also referred to as user or participant) being initially directed to a webpage (for example) that is designed to market the easy, inexpensive, or timely ability to determine effectiveness of a treatment. For instance, the webpage may advertise that, for a series of health/wellness outcomes such as sleep, pain, energy/fatigue, or cognition, multiple practical do-it-yourself treatments and trials may exist. Many dietary supplements are already consumed by individuals seeking assistance in such categories. For instance, for sleep alone the following supplements are frequently consumed: melatonin, magnesium oxide, magnesium threonate, magnesium chloride, magnesium citrate, magnesium glycinate, tart cherry, n-acetyl cysteine, l-theanine, glycine, gamma-aminobutyric acid, valerian root, apigenin, 5-hydroxytryptophan, magnolia bark, apigenin, inositol, vitamin B3, vitamin B6, vitamin B12, ashwagandha and lavender amongst others. As other examples, supplements for joint pain may include fish oil, omega fatty acids, glucosamine, chondroitin, vitamin B3, vitamin D, vitamin K, calcium, collagen, hyaluronic acid, ginger, magnesium, curcumin, and/or turmeric, whereas supplements to assist with mental focus or clarity may include vitamin B12, fish oil, CoQ10, glutamine, creatine, acetyl-l-carnatine, and/or phosphatidylserine. Scientific data on the effectiveness for each varies, and data is by no means suggestive that all individuals who would try such a material would expect to see a discernible benefit. Furthermore, any treatment incurs costs and may present risks such as unwanted side effects or adverse events. Accordingly, an individual may desire additional data to decide whether to use such a treatment.

Individual webpages for each of multiple categories of health/wellness (sleep, pain, mental acuity, and so forth) may be designed and provided both to offer general information and to route potential consumers to the list of available supplements and trials to address topics and concerns of interest to them. Subpages may subsequently describe in greater detail specific supplements. Links to other sources of data (such as third-party websites) may be available to consumers to reference existing scientific research or other information regarding use of such materials for such outcomes. Likewise, links to data regarding specific trial results available from the service provider may be made available. These and similar marketing materials may be available on the webpage and/or via other electronic or physical media, including on a smart phone app.

With reference to FIG. 1B, configuration also involves customization of a trial by the consumer/user. For example, a user may be provided with choices to customize their trial such as various available doses of material (for instance, in the case of melatonin, 12 mg, 10 mg, 5 mg, 3 mg, 1 mg, or 0.5 mg). Various dose forms may be offered, such as pills, gel caps, gummies, and so forth. Periods between dosing may be adjusted, as might periods between outcome reporting. Outcome metrics may be chosen, either or both of favorable outcome metrics and unfavorable outcome metrics, along with choosing various types of adverse event reporting. Trial duration and/or trial statistical confidence targets may also be selected and configured. In contrast with most medical scientific research, individualized N-of-1 trials in accordance with aspects of the present disclosure can better accommodate user preferences and proclivities with respect to desired speed to a result, ability to maintain compliance with a regimen, risk tolerance, and so forth.

For example, much of medical scientific research sets static thresholds for sufficient confidence so that experimental trial results are likely representative of underlying medical effects rather than a matter of statistical chance (such as with 95% confidence, or p values <0.05), whereas an individual may be satisfied using a lower confidence level. Although having long trials and high (and static) confidence may be generally advantageous across most academic and regulatory regimes, consumers may themselves face different interests regarding trial duration and statistical confidence (and the tradeoff between the two). A calculator may be integrated into a webpage (or other electronic means) to assist the user in making decisions about appropriate trial duration given a particular confidence level (or predicting a confidence level from a chosen duration).

Optionally, information may be provided to the user related to previous and relevant trial data or participant data to help the user in making trial customization selections and in deciding whether to move forward. For example, a user considering a trial to determine if melatonin helps the user sleep better may see aggregated data related to previous melatonin trials conducted by other participants that helps the user determine the duration of their own trial, dosage, outcome metrics, and so forth. Particularly useful data in making such informed decisions may be data that shows results of previous trials, which may depend on patient demographics or starting health state. Taken together, the customization options and assisting information can guide a consumer in designing a trial configuration that addresses their own goals and purposes. Once customization is finished, the consumer may be given the choice of confirming the customized trial design and proceeding to finalize a trial initiation.

If the user does not already have an account, the user will be prompted to set up an account. Account information may include limited information, restricted to only a username, password, email address, and shipping address. Alternatively, account information may further include full name, phone number, demographic information (age, sex, weight, height, race, medical information, and so forth), and credit card or other information to facilitate future transactions. When setting up an account, the user may also be provided with information related to data privacy, data use, terms and conditions for using the service, and so forth, along with prompting the user to acknowledge receiving information regarding one or more of these items and to record their consent to the terms.

The consumer may then be provided with an opportunity to pay and purchase the trial assistance service, which may initiate a contract with the trial service provider. Prior to committing to purchase, preliminary informed consent may be obtained, assuring that the consumer understands the implications of the trial, including uncertainties, possible outcomes, safety concerns, and data security, management, and rights. Alternative financial engagements may exist, such as with subscription or membership services or third-party payment systems. Upon completion of confirmed payment, the supplier may send an email or other confirmation to the consumer, which may include a link to install an app and/or a link to a dedicated web portal or other electronic resources to navigate the subsequent trial.

In parallel with supplier confirmation, the consumer may install the trial system operation code as appropriate while the supplier sends the intervention materials to the consumer. Alternatively, a consumer may obtain materials in person from a coordinating facility. Often materials may be sent in the form of a pair of bottles or containers of two types of material, a treatment and a control (for example, a placebo). In some instances, a bottle marked A may be of a placebo, and a separate bottle marked B may be of a treatment (such as melatonin or other supplement), or vice versa. In other instances, materials may be provided on blister packs, with each dose in a separate numbered (or otherwise individually identified) blister. In such an arrangement, blister packs can be made in advance, with the intervention and control in a predetermined ordering or sequence unknown to the customer, and with randomization taking place via the trial itself (“on day 1, take the dose marked #13” “on day 2, take the dose marked #8,” and so forth). Markings and schedule randomizations may be varied so that no participant would be capable of informed “guessing” of their blinded materials by seeing unblinded results from another trial.

It will be understood that, while the N-of-1 trials described herein are typically used to test a treatment against a control (for example, melatonin against a placebo), it is also contemplated to compare treatments against each other (for example, melatonin versus magnesium threonate, or melatonin at doses of 10 mg versus 3 mg versus 0.5 mg), or to compare one or more treatments against no intervention. Further combinations of materials may be tested.

Anticipating that the period between purchase and trial start may at times be lengthy, it may be appropriate to reconfirm informed consent immediately prior to trial initiation. Similarly, final details of trial configuration may be confirmed immediately prior to a trial start, such as the timing for dosing and/or outcome measurement reminders, the format and frequency of reminders, and so forth. As a final step prior to the start of the trial, the consumer may choose a start time/date. At the selected time and date, the Administration phase of the trial begins.

Referring now to FIG. 1C, during the Administration stage, the system will proceed with a progression of prompts, including when to perform treatments (for example, taking a pill during a sleep trial approximately one hour before bedtime). System prompts may take any suitable form such as an audible, visual, and/or vibration alert on a smartphone indicating it is time to take a pill (dose, treatment, or the like), and may be followed by a request from the same smartphone app to affirm if and/or when the treatment occurred. System prompts may also be in the form of SMS messages, phone calls, and so forth. Further, it should be noted that customization of detailed elements such as when a dose may be taken daily may also be integral to the method.

The time when treatment was prompted and the time when treatment was confirmed to be taken by the user may be recorded by the system. In addition, the system and methodology may employ various compliance measures to ensure and/or verify treatment. For example, electronic compliance measures may include optional methodological aids such as smart pill bottles that monitor or detect changes in the contents of the pill bottle, image capture and recognition to affirm through a smart phone camera or other means the presence of a dose on a tongue, image capture and recognition to affirm the identity of the participant by stored biometrics such as fingerprints, irises, or facial recognition, and other such compliance measures. In certain aspects of methods and systems of the present disclosure, the administering of treatments may be done through a medical device, and further may be automated or otherwise require no explicit prompting of the user.

If the consumer does not affirm treatment within an amount of time, a reminder or iterated reminders may be provided until either the patient affirms compliance or a predefined threshold for cycles of reminders has been reached (for example, after three alerts, let the participant not be reminded any further for this dose). Depending on trial protocols, a missing dose may have different consequences for the trial mechanics and/or for trial statistical analysis. Examples of trial mechanics consequences of skipping a dose include: whether or not to skip the recording of the next outcome measure (or another outcome measure paired to the skipped dose); whether or not to make up for the skipped dose using an out-of-cycle dose; whether or not to double a subsequent dose; whether or not to terminate the trial, and if the trial is continued whether or not to include the results in aggregated data; and so forth. Examples of trial statistical analysis consequences of skipping a dose include, for instance: whether or not to ignore the missed dose; whether or not to ignore a finite period of data associated with the missed dose; and so forth.

On a prescheduled, time-elapsed-since-dose, or other basis, the user may be prompted to record outcome measures and metrics. Such prompts may be similar to the prompts for dosing. A variety of Patient Reported Outcome Measures (PROMs) may be used, including metrics standardized for the particular treatment, metrics selected by the user, and/or metrics created by the user. For example, in the case of sleep-related outcome metrics, PROMs may include items typically included in a sleep diary, such as time to bed, time falling asleep, time(s) woken up, number of times woken up, perceived quality of sleep, perceived sleepiness or alertness in the subsequent period, perceived difficulty in falling asleep, and assessments of dreams. Measurement of some of these items may be facilitated by devices such as smartwatches.

In reference to FIG. 1C, outcome measures may be recorded or reported with the same frequency as doses (for example, daily), or may be recorded or reported on different cycles than that of doses (for example, pills taken multiple times per day, and outcome reporting done once per week). Outcome reporting may be done each time an outcome is prompted and/or recorded, or outcome reporting may be done as a batch of multiple recorded metrics. Outcome reporting may be done by the user themselves or by another individual (such as a spouse, parent, child, teacher, health care worker), and may involve an individual other than the user performing tests on the user to generate the metrics (for example, a parent taking a child's temperature). Outcome reporting may be manual (for example, a sleep diary), automated (for example, using a digital health tracker such as a sleep tracking device), or a combination of multiple such reporting forms. Moreover, noncompliant recording or reporting of outcome measurements may be handled in similar ways as for noncompliant dosing, as determined by the trial protocol and/or customizations.

While not separately indicated in FIG. 1C, washout periods may be used. Effects of treatments may take time to build to full effect, and to fade after a treatment course is complete. Accordingly, prior to a first block of treatments, and/or between any block of treatments, a period of time in which neither a treatment nor a placebo is used may be optionally inserted.

In general, a typical trial method may allow for many cycles (for example, days or weeks) of treatments and outcome measurements prior to reaching a threshold that signals completion of the trial. Termination may occur due to expiration of the scheduled trial duration, due to flagging of a trial for an early termination such as for noncompliance or the meeting of certain early success or failure threshold metrics, due to consumer-selected termination, and so forth. It should be noted that concluding a trial may occur on the basis of exceeding a minimum effect size that may be set by the participant or set at a medically recognized or other default level. For example, a participant may choose for a trial relating to a sleep aid will be deemed a success only if the participant gains five or more minutes of sleep per night. Termination may also be deemed to occur when an intermediate condition has been reached, for example when there is a prescheduled second stage of the trial or when a modification or adjustment to the trial becomes warranted, both of which may be based on initially designed trial conditions or based on changing trial conditions. Regardless of reason for termination, the system may then mark the trial as complete at an appropriate time. The initiation of new trials, follow-on trials, next stages of the same trial, and so forth may then commence, as appropriate, starting again at the Configuration stage.

In reference to FIG. 1D, the Completion stage may proceed upon termination of a trial. Once termination is confirmed and the system indicates that a trial is complete, the results of the trial may be made visible to the consumer. The provided results may include information in word, numeric, and/or graphical form, such as an estimate of one or more outcome measures and associated confidence intervals, a percentage likelihood of an effect size exceeding one or more thresholds, a comparison of results against other populations (including previous test participants), and so forth. At consumer selection or direction, the results may be made available to other parties, such as medical care providers, downloaded for personal use, or archived to select destinations such as an Electronic Health Record.

At this point, the packaging of the product used during the trial may be unblinded. For example, a trial service provider may provide information or means for the consumer to be able to change the product labelling to render a package that previously said “treatment or control” (for example, package B is marked as “placebo or melatonin”) to instead say simply “treatment” or “control” (for example, now package B is just marked “melatonin”). This can allow for the consumer to continue using treatment doses from that package.

In certain embodiments, a participant may qualify for a rebate (return of previously paid funds), a discount on future purchases, an account credit, and/or other incentives or rewards upon a qualifying trial completion. For instance, if the trial service provider is attempting to build sufficient data to allow submission to a regulatory body for clearance purposes, the value of a completed trial having sufficiently high compliance in dosing and outcome reporting is substantially higher than the value of a low compliance, early terminated trial. As such, a supplier may wish to subsidize and/or incent financially or by other means high participant compliance or selected trial configurations or participants.

Methods in accordance with various aspects of the present disclosure may include steps to prompt the consumer for additional purchases. For instance, in the case of a trial demonstrating an effective treatment, the participant may be prompted to purchase volumes of the treatment itself. An effective trial demonstration may warrant a follow-on trial, for example to test a lower dose of the same material, to determine whether similar benefits may be observed under different conditions that may provide the consumer with the prospect of lower risks of side effects or adverse events and/or lower cost of treatments. In the case of a trial not confirming significant effectiveness for the treatment, methods in accordance with various aspects of the present disclosure may prompt a participant for a longer trial of the same material, a higher dose trial of the same material, or a trial of an alternative treatment seeking to provide similar outcomes.

It should be noted that, due to the nature of self-administered trials, there may be doses that were late or missed, and some outcome reporting that was late or missed. This may result in studies with missing data. In certain cases, the statistical analysis can simply ignore the fact that there is missing data. In certain cases, the consumer may be notified that the data appears to be incomplete, and may be queried to determine their preference (ignore the missing data and generate statistics, invalidate the trial, extend the trial to gather more data, and so forth). One option may be to offer the consumer a biostatistical analysis service or a trial interpretation as an upsell. Under such optional services, a professional may review the results. Such human interpretation may facilitate the ability to use incomplete data sets. This option may be offered before, during, or after the trial: “The computer will run its own program for a baseline $200 trial, or for another $300, we'll have a medical professional review the results.” Such an option may be offered whether or not the trial participant fully complies with dosings and outcome recording.

Upon completion of a trial sequence, a database of aggregated or accumulated trial data may be appended. In certain embodiments, data appending may occur continuously throughout the trial, with an assessment being made later or at a final stage regarding possible use of any particular data or the database as a whole for publication on a web page, in a scholarly journal, for regulatory approval, and so forth. Efforts to remove personally identifiable information from a database may also be used to help insure patient privacy.

The present disclosure contemplates a variety of post-trial activities, such as offering and selling supplements, offering and selling new trials (for example, selling the recipient of a successful trial a new trial at a lower dose, or selling the recipient of a “failed” trial a new trial at a higher dose or of a different material), using the resulting data to update information provided to consumers as well as the available trials and trial configuration parameters, and so forth.

It will be understood from the descriptions of systems and methods herein that the individual trial participant, also referred to in various contexts as the subject, the participant, the user, and the consumer, may be a person acting on their own behalf, or may additionally encompass the efforts of a responsible person or persons acting alone or in combination to perform some or all of the tasks described herein. Likewise, a trial may be performed in a veterinary context, with a consumer acting in coordination with a pet, livestock, service, sporting, laboratory, or other animal.

It will be understood that the foregoing provides a description of methods and systems in accordance with the present disclosure that is generally applicable across various aspects and embodiments, and that those various aspects and embodiments may be employed in any suitable or appropriate combinations and permutations, whether additional or alternatively. To supplement the foregoing description, the following sections describe various aspects of methods in accordance with the present disclosure in additional detail. It will be understood that the descriptive headings are used for the purpose of organizing content, and are not meant to limit the applicability of the subject matter disclosed to any particular aspect, nor are the headings meant to signify that the aspects described thereunder are exclusive of the aspects described with respect to any other heading or embodiment disclosed herein.

### Maintaining Placebo Viability and Blinding

While unblinding of a trial upon completion has been described in reference to FIG. 1, it is also contemplated that a participant may choose for a test to remain blinded, and this selection may occur at any point prior to unblinding. Placebo (or sham) effects may be positive during a blinded trial, but would not traditionally be anticipated to be available to a participant after a trial is complete because the treatments are unblinded to the participant, and a placebos positive effects tend to dissipate after the treatment is known to be a placebo. Here, a consumer choosing for a test to remain blinded may preserve real or perceived positive placebo effect potential into the indefinite future. A participant may wish to define the trial configuration so that the results remain blinded for a finite or indefinite period upon trial completion, and perhaps even to receive (for example, purchase) further placebo material as a “treatment,” for some period thereafter.

### Unsupervised Trial Administration (Automation)

In many aspects and in many embodiments of methods in accordance with the present disclosure, the performance of certain steps or portions thereof may be partly or entirely automated from the perspective of a trial service provider. Automation may be thought of as “unsupervised,” “low or minimal manual effort,” “mostly or entirely electronic,” and so forth. Moreover, the term automated does not exclude manual completion of those actions requiring the selection, decision, or consent of a participating individual to advance through certain routine steps, for example website or smartphone app navigation, trial customization, trial ordering, informed consent, app installation, responding to dose or outcome prompts, reviewing or responding to interim and complete trial analysis, data display, and responding to invitations for further purchases. Other tasks such as material shipment may also require unique trial administration attention.

The capability of a high degree of automation across various and several aspects of methods of the present disclosure may enable a widespread use and scaling of the methods and systems described herein that can lead to minimized costs as well as maximized trial speed, convenience, and ease of administration from the perspective of a consumer. Automation can promote disintermediation (that is, the removal as mediators) of individuals who are traditionally involved in the clinical trial process, such as care providers, Institutional Review Boards (IRBs), Data and Safety Monitoring Boards, pharmacists, and even the trial service provider themselves, which may help in scaling, reducing costs, enhancing safety, and increasing speed to results for participants. Certainly, any or all of these individuals may or may not become involved before or after trials conducted in accordance with various methods described herein, as is warranted, appropriate, or desired by the participant or by the trial service provider.

### Treatments via Pharmaceuticals, Supplements or Medical Devices

While an exemplary use of the systems and methods described herein relates to trials involving dietary supplements, it will be recognized that the systems and methods are equally applicable to trials involving pharmaceuticals, medical devices, foods, beverages, or other therapeutics. It will be appreciated that trials involving a pharmaceutical or medical devices may entail additional steps to ensure safety as well as legal and regulatory compliance, likely including requirements such as prescriptions, medical licensure of a responsible overseer, and so forth. Likewise, trials of dietary supplements for outcomes other than health/wellness conditions may incur additional steps.

### Institutional Review Boards (IRBs), Exclusions and Inclusions

In some cases, it may be desirable for an IRB approval to be in place prior to initiating certain types of trials. For example, an IRB approval may be sought to cover a particular type of treatment (such as a melatonin trial for sleep), to cover a breadth of types of trials in parallel (such as multiple types of dosing, durations, confidence intervals, outcomes), or to cover multiple treatment materials (such as melatonin, magnesium threonate, and so forth) all using otherwise fairly similar protocols. In like manner, registration of trials with ClinicalTrials.gov may be warranted prior to any or all trials conducted in accordance with various aspects of methods described herein.

Traditional research methods are designed for the purposes of public data use over individualized applicability. As such, traditional trials include carefully constructed inclusion and exclusion criteria. For example, in a trial of a novel pharmaceutical, the trial may be limited only to individuals having profound or specific health concerns deemed to be candidates for treatment effectiveness. Accordingly, such a trial may have inclusion criteria (“inclusions”) for the specific health factor in question (along with possible highly restrictive thresholds for measures of that health factor) and exclusion criteria (“exclusions”) for various health factors. For consumer-directed trials such as described herein, a trial service provider may be considered as merely a facilitation tool to assist the individual in self-administration of a customized and individualized trial. Accordingly, and for certain applications, the systems and methods described herein may be conducted with minimal or effectively no exclusion or inclusion criteria. Just as an individual buying a supplement at a retail convenience store faces no exclusion or inclusion criteria, so too for an individual buying a trial for the same supplement. In instances where inclusion and exclusion criteria are employed, they may be used to screen out certain individuals. Such criteria may include one or more of: being 18 years or older; having no pre-existing medical conditions perceived by the participant as warranting medical attention pertaining to the trial; being under no medical care for health concerns related to the intended outcomes of the trial; having the ability to understand and execute the trial; and having the ability to provide informed consent.

### Peeking

Methods in accordance with certain aspects of the present disclosure may allow or make use of “peeking,” which is the revealing of certain interim data or results during a trial and before completion. Trials traditionally require participants to remain unaware of interim results to maintain scientific integrity by protecting blinding. Disclosed herein are methods that may allow participants to peek without sacrificing scientific rigor.

To allow the ability to maintain rigor after peeking, novel protocols can be used. Definitions of blinding vary, but generally refer to the prospect of isolating participants (or trial practitioners and so forth) from knowledge of which of the random treatments/controls they are receiving. A prominent benefit of maintaining their ignorance as to treatments is to minimize bias during a trial, such as when an individual expects a specific treatment to have a specific effect and then consciously or unconsciously biases the results of the trial due to that expectation. Note, however, that it may not be necessary to prevent the individual at an interim point in a trial from knowing what has transpired up until that point so long as that knowledge does not change the ability to infer what the future treatment sequence will be. Ambiguities in the use or definition of the word blinding may cloud whether the participant is actually “unblinded” or not during such a trial (if they know previous treatments, but not future). Regardless, herein is described processes by which rigor may be maintained while allowing peeking at interim results.

One element to enable peeking may be to restrict disclosure of information to treatment periods that are prior to the current period. For instance, in a four-period trial, an ABAB structure may be used, with each period consisting of a week of daily dosing and daily outcome reporting. As an example, a participant may be informed in the middle of the third period of the results of the trial up through the first two periods while minimizing the risk of losing trial rigor, because such disclosure may be done in a way that doesn't allow the participant to infer what treatment (A or B) they are about to receive (during their third period) or their future (here, fourth) period.

Another element to enable peeking is careful choice of treatment allocation structures. For instance, in a four-week trial of A versus B, 16 choices exist (A or B in each of four periods). However, most RCTs use only balanced trial structures, in which the same number of periods for a treatment and a control are used, meaning most designs omit 10 of the 16 possible options, leaving only AABB, ABAB, BABA, BBAA, ABBA, and BAAB. Further, in most designs, more than one crossover period is desired to limit confounding by slowly varying exogenous variables, further omitting use of AABB or BBAA which effectively have only one crossover due to successive repeats. Thus, ABAB, BABA, ABBA and BAAB are the most common allocation structures in four-period N-of-1 trials. Although maximizing crossover frequency is often preferred (and would favor ABAB and BABA), having designs that are symmetric in time so that slow linear changes in background exogenous variable effects can be best masked means many designers may suggest only ABBA or BAAB allocations. Unfortunately, those specific two options effectively eliminate practical peeking in most regards.

If a participant were to be told at the end of any period in a two-allocation-structure-only (that is, ABBA or BAAB) trial which treatment they just received (for instance, after the A period in ABBA, a participant found out they received A), they could instantly infer which allocation sequence they were assigned, and thus know (or guess) all future treatments they would receive. However, if all future treatment periods were completely unpredictable from any additional knowledge of past periods, then peeking may be better enabled. For instance, assume a four period A versus B protocol that is designed, non-traditionally, to include all 16 allocation sequences (including unbalanced designs). If after a first period the participant is shown that they had received A, the participant has no additional information regarding a future treatment sequence. This is because they were originally equally likely to have an AAA, AAB, ABA, ABB, BAA, BAB, BBA, or BBB conclusion to that trial (after a first period of either A or B), and now still have the same likelihoods of each sequence. No additional information has been given to the participant to allow them to better predict any future treatment sequence. Accordingly, use of the full suite of random treatment sequence allocations (rather than restricting to balanced designs, for example) may assist in enabling peeking without impairing trial rigor. The tradeoff of having imbalance and addition exogenous factor risk, may, for some individual participants and trials, may be well worth the added benefits of peeking.

Further, novel strategies to packaging may be required to be integrated into an overall protocol design. Packaging can be designed that provides doses unique to each period (for example, instead of one bottle of “A” and one of “B” for a four-period trial, four bottles, marked “1” “2” “3” and “4” that each may contain A or B depending on allocation sequence assignment). Alternatively, blister packaging or similar packaging can be used in which the blister locations of the treatments and controls are marked with specifical serial numbers with decoding known to trial administration, but not to the participant. This allows for randomization and re-randomization at any time, with the participate being prompted as to which blister from which to retrieve each dose at the time of the prompt.

Statistical analyses can be used that assess effectiveness estimates and confidence intervals at the time of peeking, much like when a trial is ended early. There has been recent development of “Anytime Valid Inferencing,” indicating that interim analysis can be performed during N-of-1 multiple crossover trials without sacrificing statistical integrity. Note that allowing for multiple periods at which a trial may be “analyzed” for completion constitutes effectively additional hypothesis testing. Testing many hypotheses during a trial (rather than just one at a traditionally scheduled completion) may increase the risk of, for instance, a false positive, and so may trigger concurrent changes in statistical thresholds to help mitigate this risk.

At the time of peeking, the participant can be made aware of any or all details regarding any element of the trial (including what was received and how their estimated results are evolving) for all periods prior to the current or imminently next dosing cycle. In certain cases, it may be preferable for peeking to be disallowed until a certain effect size is reached. It is known that different treatments can take different durations to attain expected steady state effects. For instance, an antidepressant may take weeks to reach a steady state, but a sleep aid or a topical pain relief treatment may have its entire effect present within hours or a day. As such, for a material expected to produce full effectiveness in one day, there could be one-day trial periods (for instance, one day of A, then one day of B), with single pill packaging (for example, a single dose pill pouch with a unique serial number on it), and the consumer could see the results via peeking after every single day; whereas for the material expected to have its entire effect present within weeks likely warrants having trial periods of at least those similar weeks in duration. In both cases, peeking may not be warranted until completion of incremental periods of a trial, whether that be after incremental days (period) of a topical pain relief trial, or six weeks increments (periods) of an antidepressant trial.

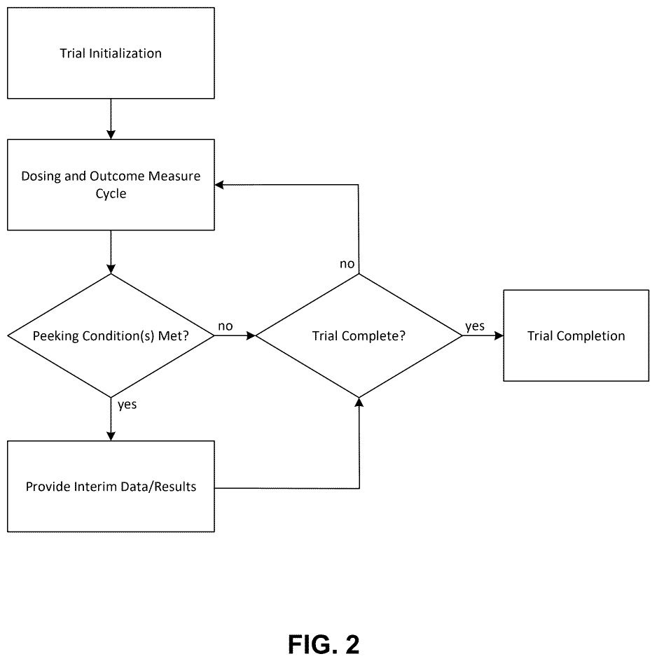

FIG. 2 depicts an example of a method flow that allows for peeking. Upon trial initialization, normal dosing and outcome measure cycles are followed, as detailed in the present disclosure. Preferably, a random period sequencing is used that facilitates maintaining blinding after peeking. Optionally, the method interrogates whether conditions have been met to allow participant peeking at interim results. For example, the peeking condition may be a set period (such as a number of days or dosing cycles) or may be based on outcome measures that indicate a surpassing a certain effect size or reaching steady state effects. If the peeking condition has not been met and the trial has not been completed, the next dosing and outcome measurement cycle is commenced. If the peeking condition is met, the participant may be provided with interim data results for any period of time that includes completed dosing and outcome measure cycles. In other embodiments, there are no peeking conditions or restrictions on peeking, which may be done as desired by the participant. The trial protocol ensures that the randomized sequencing remains hidden from the participant. At this point, if the trial completion conditions have not been met, the peeking conditions (if any) may be reset (optionally) and the next dosing and outcome measure cycle commenced. In certain embodiments, peeking may be allowed after every single period outcome measurement is reported or after every single dose/treatment is taken.

It should be understood that the techniques of offering peeking capability, including unbalanced/random treatment cycles and single dose packaging, are useful and novel methods not only in a consumer-directed, customized, decentralized context, but in general medical/health research. As such, features related to or enabling of peeking may be applicable outside of customized and/or self-administered N-of-1 trials.

### Carryover Period Estimation

In many treatment cases, single dose periods may be too short, and there may be some longer period required with multiple doses over time for full effectiveness to be observed. The expected shape of the response curve (which may, for instance, exhibit an exponential approach to an asymptotic value, if averaged across many participants to remove other sources of variability) may not be known prior to starting a commercial service, but may be estimable in the course and as a result of conducting many trials in accordance with the present disclosure. Using the specific context of single dose periods as an example, if after one day 90% effectiveness is present, 99% effectiveness is present in day two, 99.9% in day three, and so on, despite the fact that a trial of short duration may underestimate effect size, consumers may very well be interested conducting trials with even just one day periods and readily assume the risk of slight underestimation in effectiveness due to the additional speed and convenience of the shorter trial. Similarly, trials with very short periods may be particularly interesting in a context in which peeking is performed, as they may allow the consumer to see systematically (perhaps as often as every day) interim trial results, and consumers may well be willing to make sacrifices otherwise avoided in traditional trial designs (for example, unbalanced designs, underestimation of effect sizes).

Moreover, in the course of providing random sequences, there will be many cycles over which the active treatment is provided multiple periods (for example, days) in a row (for instance, in the AAAB sequence, there is a sequence of three “A” in the middle). During such repeated cycle periods, it may be possible to isolate the effects of multiple periods (for example, days or weeks) of the same treatment and thus estimate the specific per-day effectiveness values (such as 90% day 1 and 99% day 2 as described above). Over simple 4-period trials, it may be impractical to fit such data to get conclusive results, but over trials with larger numbers of periods (and with many participants in such trials), such fitting may be systematic and effective. As such, the random sequencing that helps maintain blinding after peeking can also provide treatment windows over which effectiveness profiles can be built, thereby determining carryover effect period durations (also referred to as fade-in/wash-out).

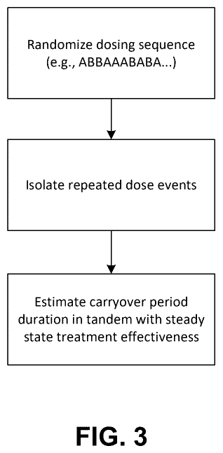

FIG. 3 indicates example steps that may be employed in estimating “steady-state” or complete effectiveness (that is, effectiveness if a treatment course were to be taken indefinitely). These steps include randomizing the dosing sequence and identifying and isolating repeated dose events of a determined length (such as at least three in a row). As mentioned, when a randomized dosing sequence is used, there will be repeated dose events, that is multiple consecutive dosing cycles in which the treatment is given. Without unblinding or revealing anything to the participant, the repeated dose events can be isolated from a data analysis perspective in order to better estimate steady state effectiveness from the measurements made during such repeated dose events. As such, the carryover period duration can be estimated in tandem with steady state treatment effectiveness.

It should be understood that the features and techniques relating to carryover period estimation are useful and novel not only in a consumer-directed, customized, decentralized context, but in general medical/health research. As such, features related to or enabling of carryover period estimation may be applicable outside of customized and/or self-administered N-of-1 trials.

### Adaptive Trials Reflecting Carryover Period Estimation

In cases where a treatment effectiveness may not have reached steady state in a first measurement during a period, alternative methods may be used. In many traditional trials, data is simply not taken or ignored at any time in which effect sizes are not stable (or are taken, and weighted “evenly” with data taken beyond that period). In accordance with the present disclosure, a statistical treatment may be advantageously used in which data taken during a transition period will be anticipated to have lower apparent effect size than later data, thereby making fits more robust. Such re-assessment of data can be made available in a variable fashion in random sequence analysis, and may allow for trials to be self-adaptive or “learning.” For instance, a trial that originally was designed to have one-week periods may be re-optimized due to learning from parallel trials (or from the same trial itself), and adjust to shorter (or longer) periods based on whether effect sizes are seen to reach steady state substantially earlier (or later) than what was assumed when originally designing the trial period.

Adaptive trials are very rarely used in traditional medical research. However, in accordance with the present disclosure, N-of-1 methods can readily accommodate adaptive trials for consumer use due to the greater flexibility and adaptability that individual participants have when experimenting on what is effective for their own health, as opposed to the traditional trial norms expected by academic or regulatory bodies.

### Co-Optimized Trials for Effectiveness Estimation in Parallel with Treatment Benefits

In accordance with various aspects, the present disclosure provides for designing a trial that may have parameters (for example, durations, confidence intervals, balance, and so forth) that vary depending on estimated effectiveness of a trial reflecting the relative importance of receiving a treatment benefit while the trial is ongoing. Further, these trial designs may adapt during a trial as new estimates on effectiveness evolve. Traditional trials often are “balanced” in that they attempt to spend equivalent amounts of time exposing a participant to a treatment and a control. If the treatment is effective, then the participant was unfortunately not receiving the possible benefit during the period when the control is administered. As such, it may be preferred to conduct alternative trials in which the benefits can be more available during the trials themselves, and/or trials complete more quickly to provide transition to a period in which benefits would be systematically available upon completion.

Trials in which an effective treatment is tested and under which more time was spent taking the treatment than the control (that is, unbalanced trials) may also be disproportionately beneficial because the participant spends more time under treatment. In accordance with methods of the present disclosure, ongoing calculations can be made prior to a trial, interior to an ongoing trial, and/or optionally in conjunction with data from parallel and/or preceding trials, to adjust the relative ratio (balance) of treatment exposure versus control exposure (or versus other treatments) to thereby co-optimize the benefit of completing a trial early, of having rigorous results for that trial, and of optimizing the time ratio an individual receives positive treatments instead of a placebo. Further, rather than a system “imposing” an imbalance, a consumer may be given the choice to design an imbalance (either by suggestion, or by their simple whim).

### Aggressive Customization

In accordance with various aspect of methods disclosed herein, there may be opportunities for a consumer to specify elements of trial customization that are not pre-defined by a supplier. While a supplier may have a necessarily limited inventory of candidate trials, from the consumer perspective there are myriad choice, including something like 90,000 different commercial dietary supplements (as one example). As such, it is foreseeable that a consumer may wish to design and conduct a trial on a non-stocked material.

In certain aspects of methods of the present disclosure, a consumer may select a supplement (for example) for testing that the trial supplier does not normally stock or sell. In such an instance, the consumer may, for example, direct the trial supplier service party to a third-party online retail supplier of the desired supplement, and initiate a trial on such a supplement. The trial supplier could directly buy (retail) a trial amount of that supplement, repackage it (for example into new bottles, into single dose or other easily blinded delivery vehicles), provide a “matching” placebo, and deliver them to the consumer as a placebo blinding service. Alternatively, a consumer may send in a material and/or placebo, and a trial service provider may provide simply a blinding service. The trial service provider may further provide assistance in determining an appropriate protocol (how many days, what outcomes to measure, and so forth), and offer the same or similar software/statistics package used for their standard offerings. Alternatively, the consumer may choose their own outcome metrics, such as defining a question to be asked every day during a trial along with a ratings scale (for example, “How refreshed were you upon waking this morning on a scale of 1 to 5?”).

### Mid-Trial Adaptability

Traditional clinical trials are largely static, having only limited deviations from pre-existing dosing schedules. For single consumer trials such as those useful with systems and methodologies in accordance with the present disclosure, many consumer trial parameter choices such as duration, confidence level, importance of treatment/results (that is, trial imbalance), may engender reasons to change a treatment schedule. Particularly for cases where single dose packaging is provided, rather than forcing a consumer to stick with dosing to the predefined schedule (for example, after the first three days ABB of an anticipated ABBAAABABA schedule), the process may update with a new assignment (such as ABBAABBAABBAABB) having new parameters for balance, duration, period, and so forth. Trials may also adapt (with or without consumer awareness) based on statistical observations made by the system. For example, adapting from a balanced trial to an imbalanced trial with more active exposure may be warranted to expedite trial conclusion when extremely low variability in outcome measurements is seen for placebo.

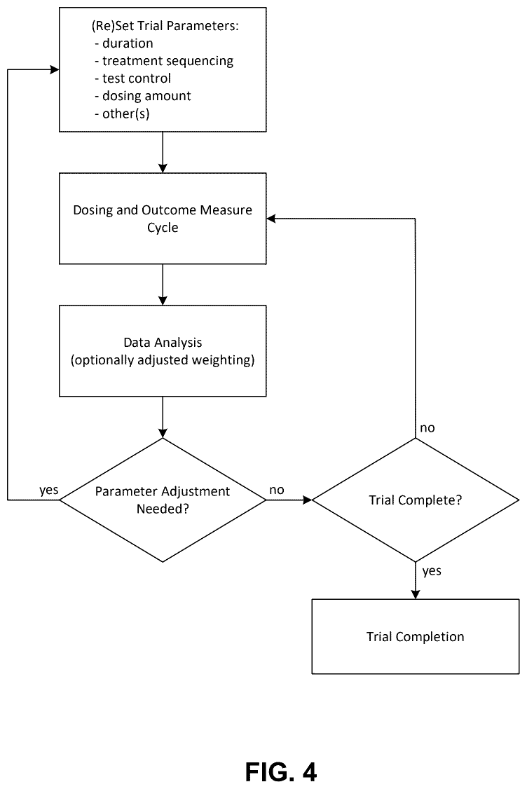

Aspects of the various trial adaptation techniques discussed herein may be summarized with reference to FIG. 4. Initially, the trial parameters may be set through customization and/or predetermined (for example, based on supplier selections or based on similar previous trials) or default selections. Dosing and outcome measure cycles are then commenced. As data is gathered, it is analyzed to determine if one or more of the trial parameters need adjustment, for example in accordance with descriptions provided herein related to reducing or extending trial duration, making a balanced trial unbalanced, adjusting the dosing amounts, adjusting the test control (such as substituting a different treatment for the placebo), and so forth. In addition, the data analysis can be adjusted to weight outcome measures differently to account for the adapted trial parameters or consumer preferences. For example, the initial configuration of a trial relating to a sleep aid supplement, the outcome measures of hours slept and number of times awoken may be designated, with hours slept being the primary outcome of interest. Post adaptation, the participant may decide to redesignate the number of times awoken as the primary outcome of interest.

### Consumer Trials as a Claim Validating Technique

Changing a label on a supplement to allow claiming of specific benefits anticipated from taking that supplement requires a body of data to substantiate that claim to the FDA. In methods described herein, consumers who are interested in the trial data for their own purposes may also allow access to and permission for the trial supplier to use the same data. With sufficient volume of such data, it may be possible to collate and meta-analyze the data to substantiate sufficient conclusory value to thereby assert a general prospective benefit (for example, a prospective claim that a particular supplement assists with minor joint pain). In accordance with aspects of the present disclosure, methods include using consumer self-administered N-of-1 trials to build documentation in support of subsequent demonstration of benefit to external bodies, including on the trial service's own webpage, peer reviewed scientific publications, and to FDA or regulatory bodies. Posting the results on the trial service provider's webpage may help advise others on applicability of a trial, and may also be performed in a fashion allowing independent download or consumer-data-analysis off-line.

### Pharmacogenomics

In addition to providing the consumer with the data, researchers may find value in the data. The value of the sourced data may grow substantially if participants source additional data into the process. Consumers may add basic data (for instance, age, sex, weight, height, race, and so forth) or medical data (such as conditions, values of specific laboratory data) or even sophisticated data (such as DNA sequencing data). The combination of DNA data paired with treatment effectiveness may be useful in developing or sourcing pharmaceutical treatments. As such, methods of the present disclosure include using consumer N-of-1 data in combination with other sources of demographic or medical data to assist in medical treatment development and/or medical sales.

### Moving Baseline Treatments

Traditionally, N-of-1 crossover trials are done on treatments that have transitory effects. In other words, the participant's health state entering each period (perhaps after a washout interim period) must be the same upon entering all other periods. This means that traditional N-of-1 trials are best suited to treatments of symptoms of underlying chronic conditions (such as joint pain for osteoarthritis) rather than treatments that may tend to cure a condition or place the participant in a different health state (such as treatment of an infection with an antibiotic). Under such conditions, N-of-1 trials would not be of use for a variety of health/wellness concerns of interest to consumers such as weight loss for overweight individuals or weight gain for athletes. If a dietary supplement aided in weight loss, a normal N-of-1 statistical analysis would be invalid since after each active treatment period the participant would be expected to enter the next period of the trial having a lower weight, and the lower weight would be anticipated to have a different treatment response (it is likely easier to lose weight when heavier).

In accordance with aspects of the present invention, it is recognized that evolving heath/wellness states of an N-of-1 trial participant are addressable by fitting the shape of each new outcome response curve to each outcome/treatment condition. Restated, N-of-1 trials can continue to be performed on evolving baseline conditions (or “cures”) utilizing newly acquired data. While the presence of additional curves to be fit may add complication and risk in data interpretation (and thus reduce the perceived scientific research value of moving baseline trials), those risks may be outweighed by the usefulness for consumers in providing customizable and personalized trials for a whole other class of treatment types that may tend to produce improving health conditions. Such work would have been disfavored and/or impractical in a traditional large-population trial research where it would be easier to avoid the problem and rely on non-crossover trials. However, in consumer-directed trials, the value to the consumer is so high, and the potential errors are of such relatively small consequence, that crossover-trials on evolving baseline conditions become particularly useful and practical. As such, in accordance with various aspects, methods of the present disclosure include using N-of-1 consumer trials on moving baseline treatments, and optionally to address baseline shifts with additional statistical terms to fit shift effects and maintain full statistical rigor.

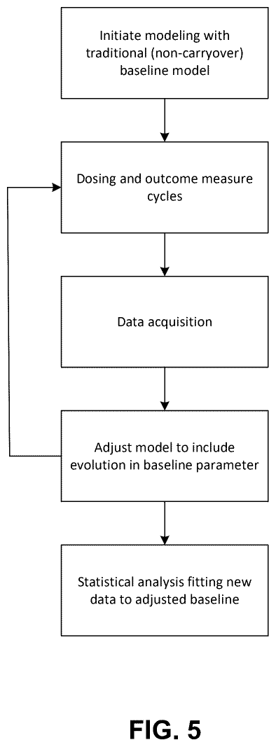

FIG. 5 schematically illustrates a method flow that may be used in conjunction with moving baseline studies as described herein. In the case of weight loss, for instance, it may be possible to start a trial without any presupposition of the effect of changed weight on weight-loss sensitivity. However, as data accumulates both from the trial of one individual, and from the trial of preceding or contemporary participants (or from general public literature), a more refined model may be developed to estimate effectiveness of weight loss as a function not only of treatment but also of evolving participant weight (whether in isolation or in comparison to initial weight pre-trial). Such a more refined model may be used to update effect size (for example, not “supplement A after 4 week-long periods appeared to help induce weight loss in individual X of 4 pounds,” but “supplement A after 4 week-long periods appeared to help induced relative weight loss in individual X of Y % of existing weight”).

It should be understood that the features and techniques relating to moving baseline treatments are useful and novel not only in a consumer-directed, customized, decentralized context, but in general medical/health research. As such, features related to or enabling of moving baseline treatments may be applicable outside of customized and/or self-administered N-of-1 trials.

### Cross-Trial Meta-Analysis and Data Leverage

In traditional RCTs, results from multiple trials may be collated and meta-analyzed. However, in most such cases, the trials involve extremely similar treatments and outcomes. Herein are disclosed alternative methods in which trials may vary more broadly yet offer uniquely useful information in guiding other trial designs or interpretation. In a consumer-facing trial administration service, large volumes of parallel data for somewhat similar trials will emerge. For instance, in certain circumstances, many consumer-participants may have executed trials for improved joint pain using various dietary supplements such as turmeric, fish oil, glucosamine-chondroitin, and so forth. All may have used identical pain reporting measures, dosing schedules, and perhaps even identical placebos or other trial parameters.

Design of a new trial (either for an individual, or as a protocol for a new supplement altogether), may make use of anticipated knowledge of how such trials are likely to evolve based on prior trial results. As a specific example, a consumer having just completed a trial using turmeric in an attempt to assist with pain but failing to see a benefit from turmeric may wish to immediately start a new experiment on fish oil to assist with pain. The observations of the daily variability in pain during the placebo portions of the former trial may be anticipated to be highly predictive of the daily variability of the future trial and may thus help in design of the trial structure (how long, what period structure, and so forth), or in actual variability (and mean) calculation themselves.

Further, a series of trials on an individual may be structured so as to be contiguous and overlapping. A “BAAB” trial with B as a control may follow with a second “BCCB” trial with B as a control and C as a new treatment. The last B period and the first B period may actually be the same time period (that is, the two trials become BAABCCB). Such strategies are described herein as being conducted on an individual participant basis, but population level estimates may also be of use in broader trial execution as well, particularly in a Bayesian, meta-analysis context. It should be understood that the features and techniques relating to cross-trial meta-analysis and data leverage are useful and novel not only in a consumer-directed, customized, decentralized context, but in general medical/health research.

It should be understood that any section headings provided herein are for the sole purpose of readability, and the features described under specific headings may be used in combination with features described elsewhere in the present disclosure. Moreover, it should be understood that several features disclosed herein may be useful whether or not they are employed in a customized and/or self-administered N-of-1 trial.

It should be understood that various aspects disclosed herein may be combined in different combinations than the combinations specifically presented in the description and accompanying drawings. It should also be understood that, depending on the example, certain acts or events of any of the processes or methods described herein may be performed in a different sequence, may be added, merged, or left out altogether (for example, all described acts or events may not be necessary to carry out the techniques). In addition, while certain aspects of this disclosure are described as being performed by a single module or unit for purposes of clarity, it should be understood that the techniques of this disclosure may be performed by a combination of units or modules.

All scientific and technical terms used herein have meanings commonly used in the art unless otherwise specified. The definitions provided herein are to facilitate understanding of certain terms used frequently herein and are not meant to limit the scope of the present disclosure.

As used herein, the term “configured to” may be used interchangeably with the terms “adapted to” or “structured to” unless the content of this disclosure clearly dictates otherwise.

As used herein, the term “or” refers to an inclusive definition, for example, to mean “and/or” unless its context of usage clearly dictates otherwise. The term “and/or” refers to one or all of the listed elements or a combination of at least two of the listed elements.

As used herein, the phrases “at least one of” and “one or more of” followed by a list of elements refers to one or more of any of the elements listed or any combination of one or more of the elements listed.

As used herein, the terms “coupled” or “connected” refer to at least two elements being attached to each other either directly or indirectly. An indirect coupling may include one or more other elements between the at least two elements being attached. Further, in one or more embodiments, one element “on” another element may be directly or indirectly on and may include intermediate components or layers therebetween. Either term may be modified by “operatively” and “operably,” which may be used interchangeably, to describe that the coupling or connection is configured to allow the components to interact to carry out described or otherwise known functionality.

The singular forms “a,” “an,” and “the” encompass embodiments having plural referents unless its context clearly dictates otherwise.

As used herein, “have,” “having,” “include,” “including,” “comprise,” “comprising” or the like are used in their open-ended sense, and generally mean “including, but not limited to.” It will be understood that “consisting essentially of,” “consisting of,” and the like are subsumed in “comprising,” and the like.

Reference to “one embodiment,” “an embodiment,” “certain embodiments,” or “some embodiments,” and so forth, means that a particular feature, configuration, composition, or characteristic described in connection with the embodiment is included in at least one embodiment of the disclosure. Thus, the appearances of such phrases in various places throughout are not necessarily referring to the same embodiment of the disclosure. Furthermore, the particular features, configurations, compositions, or characteristics may be combined in any suitable manner in one or more embodiments.

The words “preferred” and “preferably” refer to embodiments of the disclosure that may afford certain benefits, under certain circumstances. However, other embodiments may also be preferred, under the same or other circumstances. Furthermore, the recitation of one or more preferred embodiments does not imply that other embodiments are not useful and is not intended to exclude other embodiments from the scope of the disclosure.

What is claimed is:

1. A kit for conducting a blinded N-of-1 trial, the kit comprising a first container that includes a first treatment material or a control material but not both, wherein the control material comprises a placebo or a second treatment material, and wherein the first container includes a label uniquely identifying the first container and indicating that the first container contains either the first treatment material or the control material and listing ingredients of the first treatment material and the control material.

2. The kit of claim 1, further comprising a second container that includes the other of the first treatment material and the control material that is not contained in the first container, the second container including a label that uniquely identifies the second container and indicating that the second container contains either the first treatment material or the control material and listing ingredients of the first treatment material and the control material.

3. The kit of claim 1, wherein the first container includes at least one multi-dose bottle.

4. The kit of claim 1, wherein the first container comprises a plurality of single-dose units arranged in a unit-dose blister card matrix.

5. The kit of claim 2, wherein the first container and the second container each comprise a plurality of individual single-dose sachets.

6. The kit of claim 1, further comprising instructions associated with the first and second containers, the instructions specifying, according to a blinded treatment regimen, (i) a dosing schedule for administering doses from the first and second containers to an individual, and (ii) a response-recording schedule for recording responses of the individual relating to one or more outcome measures.

7. A kit for conducting a blinded N-of-1 trial, the kit comprising:
a treatment material packaged in a first packaging unit and a control material packaged in a second packaging unit, wherein the control material comprises a placebo or a second treatment material; and
a kit label indicating that the kit contains the treatment material and the control material and that lists the full ingredients of the treatment material and the control material but that does not indicate which of the first packaging unit and the second packing unit contains which of the treatment material and the control material.

8. The kit of claim 7, wherein the first and second packaging units include respective first and second packaging unit labels, the first and second packaging unit labels each having a unique packaging unit identifier and indicating that the respective packaging unit contents includes one of the treatment material or the control material.

9. The kit of claim 7, wherein the first packaging unit and the second packaging unit each comprise at least one multi-dose bottle.

10. The kit of claim 7, wherein the first packaging unit and the second packaging unit each comprise a plurality of single-dose units arranged in a unit-dose blister card matrix.

11. The kit of claim 7, wherein the first packaging unit and the second packaging unit each comprise a plurality of individual single-dose sachets.

12. The kit of claim 10, further comprising instructions associated with the first and second packaging units, the instructions specifying, according to a blinded treatment regimen, (i) a dosing schedule for administering doses from the first and second packaging units to an individual, and (ii) a response-recording schedule for recording responses of the individual relating to one or more outcome measures.

13. A kit for conducting a blinded N-of-1 trial, the kit comprising:
a plurality of single-dose packages, each single-dose package containing a dose of either a treatment material or a control material;
a respective identifier associated with each single-dose package, wherein the respective identifiers distinguish the single-dose packages from one another without identifying to a participant whether any particular single-dose package contains the treatment material or the control material; and
instructions associated with the plurality of single-dose packages, the instructions specifying a randomized dosing schedule that identifies, for each of a plurality of dosing times, one of the respective identifiers corresponding to a single-dose package from which the participant is to retrieve a dose,
wherein a decoding of the respective identifiers to the treatment material or the control material is maintained by trial administration and hidden from the participant during at least a portion of the blinded N-of-1 trial.

14. The kit of claim 13, wherein the plurality of single-dose packages comprises a blister pack having a plurality of separately identified blister locations.

15. The kit of claim 13, wherein the plurality of single-dose packages comprises a plurality of single-dose pill pouches.

16. The kit of claim 13, wherein the randomized dosing schedule is configured so that disclosure of one or more previously administered doses does not identify one or more future doses to be administered according to the randomized dosing schedule.

17. The kit of claim 13, wherein the instructions further specify a response-recording schedule for recording responses of the participant relating to one or more outcome measures.

18. The kit of claim 13, wherein the instructions are configured for use with an electronic user interface that prompts the participant to retrieve a dose from a single-dose package corresponding to one of the respective identifiers.

19. The kit of claim 13, wherein the control material comprises a placebo or a second treatment material.

20. The kit of claim 13, wherein the dosing schedule is randomized such that the participant is unable to determine, based on the first and second blinded identifiers, whether a future dose corresponds to the treatment material or the control material.
